(19) United States
(12) Patent Application Publication
(10) Pub. No.: US 2005/0288769 A1
(43) Pub. Date: Dec. 29, 2005

(54) Mechanical structures and implants using said structures

(76) Inventor: Oren Globerman, Kfar-Shmaryahu (IL)

Correspondence Address: WOLF, BLOCK, SCHORR & SOLIS-COHEN LLP, 250 PARK AVENUE, NEW YORK, NY, 10177, US

(21) Appl. No.: 10/517,940

(22) PCT Filed: Jun. 12, 2003
(86) PCT No.: PCT/IL03/00503

§ 371 Date: Jul. 18, 2005

Related U.S. Application Data
(60) Provisional application No. 60/387,930, filed on Jun. 13, 2002.

(51) Int. Cl. A61F 2/06

(52) U.S. Cl. 623/1.15

(57) ABSTRACT

A deformable medical implant, comprising: a body defining at least two anchor points, which body is adapted to be deformed so that the two anchor points are moved relative to each other; at least two elongate extensions, each extension fixed to one anchor point; a bridge coupling at least two of said extensions to each other; and at least two hinges defined on at least one of said extensions, two of said at least two hinges having different preferred bending directions and being defined on one extension.

### RELATED APPLICATION

This application claims the benefit under 119(e) of 60/387,930 filed Jun. 13, 2002, the disclosure of which is incorporated herein by reference.

### FIELD OF THE INVENTION

The present invention relates to the field of mechanical structures and especially structures that couple tension force in one plane to force in another plane.

### BACKGROUND OF THE INVENTION

A main cause of death and disability in the modern world is the stenosis of blood vessels. Left unchecked, such stenosis can cause degeneration of tissues, such as the kidneys or the brain or even the acute failure of a tissue. For example the heart or the brain may experience an acute failure if such a stenosed vessel spasms or is clogged by floating debris (which debris is often fallout from a different stenosis).

A common procedure for treating such stenosis includes expanding a stent inside the stenosis vessel. The stent has an inner lumen sufficient for unobstructed flow of blood therethrough and is strong enough to prevent collapse of the vessel onto the lumen.

Often, however, the stenosis is at a branching in a blood vessel, for example, in the coronary vessels where they connect to the aorta. Placing a standard stent in a branch is difficult if not impossible. If the stent does not reach the branch, the point of branching will remain narrowed. If the stent extends beyond the branch, an obstruction is formed. This obstruction may cause turbulence and/or interfere with a second catheterization. As catheterization typically involves sliding a catheter along the aorta, the catheter tip will bump against or go around the protrusion (and the coronary vessel) if the stent extends beyond the branch.

U.S. Pat. Nos. 5,607,444 and 5,868,777, the disclosure of which is incorporated herein by reference, teaches a stent with a flared section. The flared section, actually a plurality of fingers, apparently either flare by themselves or are flared using a special balloon, to engage the branching area outside of the vessel in which the rest of the stent is implanted.

Various mechanisms have been described for causing parts of a tubular structure to project outside of a surface of the structure. WO 00/44319, for example, the disclosure of which is incorporated herein by reference, describes patterning a tube with slits and weakened areas so that when the tube is compressed axially, a plurality of protrusions formed from the tube surface with project in a general radial direction. However, this device is not a stent. WO 99/62415, the disclosure of which is incorporated herein by reference, describes various mechanism for extending spikes in a radial direction relative to a tube used as an anastomotic connector. In one described embodiment, the device is made of a super elastic or shape memory material and the spikes are trained to point out, when the device is released.

### SUMMARY OF THE INVENTION

An aspect of some embodiments of the invention relates to a mechanism for controlling the distortion of a structure, in which tension forces in a device plane of an implanted device cause a portion of the device to exit the device plane. In an exemplary embodiment of the invention, the control is achieved by changing the direction of the smallest cross-section moment of inertia at sections of the device, for example, providing one or more of voids, weakening, thining, thickening and/or stiffening in a manner that is not symmetric relative to the device plane, for example, oblique cut-outs and/or voids that do not necessarily connect two opposing surfaces of the device. In an exemplary embodiment of the invention, the non- symmetric moment of inertia makes the device bend out of plane, when suitable in-plane forces are coupled to the device and cause bending at the sections with changed cross-section moment of inertia.

The term “device plane” is used herein in a mathematical sense of a two dimensional shape (but not necessarily flat) that conforms to the surface of the device, assuming the device were of zero thickness. As the device has some thickness, the term device plane also includes the general volume that is between an inner and outer surface of the device. In the example of a cylindrical stent, this volume corresponds to the material that would comprise the stent if it were a solid tube.

In an exemplary embodiment of the invention, the device is formed of a thin material, such as sheet metal. It should be appreciated that in cylindrical devices, the device plane is the surface of a cylinder. In some cases, the cross-section is polygonal, so the device plane is piece-wise flat. It should also be noted that in devices formed by cutting sheet metal, the device plane is locally parallel to a flat plane that is tangential to the device surface.

In an exemplary embodiment of the invention, the mechanism is provided as a pair of mirror-symmetric elements, connected at one end and assisting in guiding the force transformation in a desired direction. However, non-symmetric elements may be used, or there may be only one bendable element attached to an element with no specific bending points defined on it. In an exemplary embodiment of the invention, the elements bend in a direction of minimum moment of inertia. By proper design of the bending direction of each element (e.g., the moment of inertia at the bending locations), a certain degree of control may be achieved on the bending of the elements, in particular, out of plane bending.

In an exemplary embodiment of the invention, the modifications function as hinges, which, being functionally oblique to the device plane, couple forces in the device plane to directions outside of the device plane. In the example of a stent, when the stent is expanded radially, two fingers that are attached at their tips have their bases pulled apart. This applies a stress on the fingers. Assume that the fingers have a shape of a square beam, with the modifications comprising a wedge shaped cut, with a base on one side of the beam (and parallel to the plane) and an apex at another side thereof. The hinge, being weaker than the rest of the finger (relative to the applied moment) will tend to bend. However, being oblique, its bending includes an out of plane component, which causes the fingers to bend away from the stent plane. In an exemplary embodiment of the invention, by providing two or more such modifications and/or movement limiting elements the net direction of distortion can be controlled to have a significant component out of the plane, with other distortion directions being minimal and/or compensated for.

Various characteristics of the modification may be set, in order to achieve desired results. For example, the depth of a void may be used to define the amount of force required to cause a bend at the void. A width of a void may be used to define how far the bending can progress before the edges of the void meet and further bending is prevented.

In an exemplary embodiment of the invention, a plurality of modifications that change the resistance to bending are provided in straight or uniformly curved sections of an un- deployed device, rather than at comers or sharp bends thereof. However, it is noted that in some embodiments of the invention, the modified portions may become corners when the device is deployed. In an exemplary embodiment of the invention, the location, size, orientation and/or other properties of the weakening(s) are selected to positively control where the device will bend or otherwise distort when it is deployed. Alternatively or additionally, the properties are selected in order to positively control an order in which different parts of the device distort (e.g., which hinge will bend first can depend on the relative moment of inertia and applied forces to each hinge). Alternatively or additionally, the properties are selected to limit one or more aspects of the distortion and/or to define a final distorted configuration of a deployed device. In some exemplary embodiments of the invention, the distortion comprises distortion in the device plane and out of the device plane. One or both are optionally controlled by selection of the modification locations.

In an exemplary embodiment of the invention, the structure is a cylindrical mesh-like structure, for example a stent. However, some embodiments of the invention are practiced using non-mesh elements and/or for non-stent devices. A particular property of stents and similar devices is that they are typically radially expanded during deployment. In an exemplary embodiment of the invention, this radial expansion is utilized for providing the above mentioned in-plane forces.

In an exemplary embodiment of the invention, a part of the structure is distorted to be angled at 25°, 45°, 80°, 90°, 120°, 150° or any smaller, intermediate or greater angle relative to the device plane. These angles are exemplary of various bifurcation angles found in various blood vessels and/or other hollow organs in the body.

In an exemplary embodiment of the invention, the modification (e.g., void, weakened area) is formed at an oblique angle relative to the device plane. Alternatively, the modification is perpendicular to the device plane. Optionally, the sides of a void are used to define a limit on distortion, to be reached when such a void is distorted enough so its sides contact each other.

In an exemplary embodiment of the invention, the modifications comprise voids. Optionally, the voids comprise slots with a continuously varying depth, for example, at a fixed angle to the plane. Alternatively or additionally, the voids comprise slots sections with fixed depths. Slot sections of different depths may be located side by side to approximate an inclined slot. In a plane parallel to the device plane, the slots may be, for example, straight, curved or symmetric (e.g., round). The slot may lie in a plane perpendicular to or oblique to the device plane. Alternatively or additionally, at least some slots do not wholly lie in a single plane. The slot may have a fixed or a varying profile geometry, for example, a pyramid or a cylinder shaped void may be provided. In some embodiments of the invention, a slot is formed in straight, elongate section of the device, along which tension forces are applied. Such a slot is optionally in a plane perpendicular to the direction of the tension forces. Alternatively, the plane of the slot may be aligned based on the desired distortion. Similar geometrical considerations may be applied to weakenings and stiffenings (e.g., the shape of the modification).

In some embodiments of the invention, a single slot is approximated by a series of slots, voids and/or slot sections, for example the series approximating a slot shape and/or a slot orientation.

Optionally, a stiffening treatment or a thickening or widening of the device is provided to prevent bending at locations and/or in directions where bending would otherwise be facilitated by the provision of the modifications.

In an exemplary embodiment of the invention, the modification is provided in an inner part of the hinge defined by the modification. Alternatively or additionally, the modification is provided in an outer part. It should be appreciated that the direction of bending of a hinge is determined, in part by the modification symmetry. For example, if from a square cross-section two triangles with bases that are opposing and parallel to a diagonal of the square are removed, the preferred bending direction may depend on the symmetry of the profile relative to the diagonal, for example, with bending preferred towards the direction which has a smaller moment of inertia (e.g., has a smaller cross-section). Alternatively or additionally, the bending direction may depend on the direction of application of force and/or on other modifications made in the structure, which, for example also induce out of plane distortion.

In an exemplary embodiment of the invention, the device is designed to have a limited (e.g., at least for a particular applied force) extension of the non-planar sections. In an exemplary embodiment of the invention, the modified areas have a bending profile that is stepped, so that once a certain bending and/or twisting is achieved, further bending cannot take advantage or takes lesser advantage of the modification, thus preventing or reducing further out of plane distortion. Alternatively or additionally, one or more struts or wires are provided to limit the relative movements (e.g., away and/or towards each other) of points on the device, thus limiting and/or otherwise controlling out of plane motions. It should be noted that a series of modified areas may be used, each of which may each define one part of a bending profile, so that as a unit they define a complex bending profile.

Optionally, the device comprises a plurality of parts that are moved off the device plane by the methods described above. The movements of these parts may be coupled and/or restricted to each other, for example, causing one part to distort less if another part distorted more and/or controlling the relative direction of distortion. Alternatively, one or more independent distorting parts may be provided.

In an exemplary embodiment of the invention, a stent is provided having a cylindrical stent portion and a flaring portion. This stent is optionally used in a branching vessel stenosis situation, especially if the branch is near perpendicular. The cylindrical section goes into the branching vessel and the flaring portion caps the bifircation area in the main vessel. Optionally, the geometry of the flaring section (but not its orientation) is fixed using coupling between the out-of-plane motion of various parts thereof. Optionally, the flaring portion defines a plurality of fingers. Alternatively or additionally, the flaring portion defines a mesh. The stent may be, for example, plastically, elastically, super-elastically deformed and/or use a shape memory mechanism. Optionally, the degree of flaring is limited to defining a cap having an internal angle of less than 180, for example, 170° or 160°, or any intermediate or smaller angle. This limitation may be useful to prevent strain on blood vessels in some anatomical and/or physiological situations.

Optionally, or alternatively, out of plane portions of a stent are used to anchor the stent. Optionally, the out of plane structures are provided at a point in the stent away from its end, for example at its middle, for example, to define one or more protrusions. Alternatively or additionally, the mechanism is used to provide a bend in the stent during deployment. Alternatively or additionally, the structures are not radially symmetric with respect to the stent axis.

An aspect of some embodiments of the invention relates to controlling deformation of a medical implant. In an exemplary embodiment of the invention, modifications are provided in a manner that is not symmetric to a device plane nor to an axis of a structural element part being modified. Optionally, when the device is expanded (or, for some devices, two parts of the devices are brought together or brought apart), these modifications cause the extension or twisting outwards of, for example, one or more barbs or flared segments suitable for anchoring the device, for example a stent.

An aspect of some embodiments of the invention relates to an advance limiter for use in an ostial stenting. In an exemplary embodiment of the invention, a stent is positioned in a branching side vessel using a catheter. In an exemplary embodiment of the invention, the catheter and/or the stent include an advance limiter that assists in positioning of the stent in a desired axial position relative to the branch. In some embodiments of the invention, the stent expands in an oblique manner and/or flares in an oblique manner. In an exemplary embodiment of the invention, when the advance limiter is deployed, the catheter turns so that a known angular sector of the catheter is in the direction of minimum (or maximum) bend of the catheter. This may be done, for example, by a balloon advance limiter, which has an oblique back end that is axially aligned with the bending location of the catheter, so that when the balloon is inflated, it causes the catheter to rotate until the bend location is properly aligned with, for example, a notch in the balloon.

In an exemplary embodiment of the invention, the stent is at least partially expanded before it is advanced all the way, such that a flaring part of the stent increases in radius enough to assist in positioning the stent. Optionally, the stent is then expanded more, which may or may not affect the degree of flaring of the flaring portion, depending on its design. Alternatively or additionally, the catheter comprises a balloon or an extendible structure of another kind that, when extended, prevents axial advance into the side vessel beyond a desired amount.

An aspect of some embodiments of the invention relates to a mesh stent having at least one end that is more readily expandable than a center and other end of the stent. In an exemplary embodiment of the invention, the mesh is designed to allow it to expand enough to flare out to about or more than 90 degrees relative to an axis of the stent. In an exemplary embodiment of the invention, when the stent is expanded using a single long balloon, the flaring portion expands first, for example, when a first balloon pressure is reached and the rest of the stent expands when a second balloon pressure is reached. In an exemplary embodiment of the invention, the mesh sections of the stent comprise coiled or folded sections (e.g., folded in the device plane), to allow for a relatively greater expansion of the flaring portion.

There is thus provided in accordance with an exemplary embodiment of the invention, a deformable medical implant, comprising:
a body defining at least two anchor points, which body is adapted to be deformed so that the two anchor points are moved relative to each other;
at least two elongate extensions, each extension fixed to one anchor point;
a bridge coupling at least two of said extensions to each other; and
at least two hinges defined on at least one of said extensions, two of said at least two hinges having different preferred bending directions and being defined on one extension. Optionally, said two elongate extensions each comprise a plurality of hinges. Optionally, the hinges on said one elongate extension are a mirror of the hinges on the other, coupled, extension.

Alternatively or additionally, the hinges on said one elongate extension have different axial locations than corresponding hinges a second, coupled, elongate extension.

In an exemplary embodiment of the invention, at least one of the hinges on said one elongate extension has a hinge bending direction different from corresponding hinges a second, coupled, elongate extension.

In an exemplary embodiment of the invention, at least one of the hinges on said one elongate extension has a resistance to bending different from corresponding hinges a second, coupled, elongate extension.

In an exemplary embodiment of the invention, only one of said at least two elongate extensions comprises a plurality of hinges.

In an exemplary embodiment of the invention, at least two of said plurality of hinges have bending axes that are oblique to a device plane of said body, said device plane being a substantially two-dimensional mathematical surface conforming to the general geometry of the device.

In an exemplary embodiment of the invention, at least one of said plurality of hinges has a preferred bending direction in a device plane of said body, said device plane being a substantially two-dimensional mathematical surface conforming to the general geometry of the device. Optionally, at least one of said plurality of hinges has a preferred bending direction perpendicular to a device plane of said body, said device plane being a substantially two- dimensional mathematical surface conforming to the general geometry of the device.

In an exemplary embodiment of the invention, said hinges are arranged to cooperate with said bridge to bend said extensions in a direction including a component perpendicular to a device plane of said body, when said anchor points are moved apart, said device plane being a substantially two-dimensional mathematical surface conforming to the general geometry of the device. Optionally, said hinges are arranged to bend at least one of said extensions at at least two points, in different directions.

In an exemplary embodiment of the invention, said hinges are arranged to bend said extensions at least 45 degrees away from said device plane. Optionally, said hinges are arranged to bend said extensions at least 80 degrees away from said device plane. Optionally, said hinges are arranged to bend said extensions at least 90 degrees away from said device plane. Optionally, said hinges are arranged to bend said extensions at least 120 degrees away from said device plane.

In an exemplary embodiment of the invention, at least one of said hinges comprises cuts in said extension. Alternatively or additionally, at least one of said hinges comprises a weakening in a position along said extension. Alternatively or additionally, at least one of said hinges comprises a bore in said extension. Alternatively or additionally, said extensions extend axially away from said body, prior to moving apart of said anchor points.

In an exemplary embodiment of the invention, said extensions extend axially towards said body, prior to moving apart of said anchor points. Alternatively or additionally, said bridge is defined at an end of said extensions. Alternatively or additionally, said bridge is deformable. Optionally, said bridge is more resistant to bending than said hinges.

In an exemplary embodiment of the invention, said hinges are plastically deformable. Alternatively or additionally, said plurality of hinges comprise at least three hinges on a single extension. Alternatively or additionally, said body is cylindrical. Alternatively or additionally, said implant is adapted for implanting in a blood vessel.

In an exemplary embodiment of the invention, said implant is a stent. Optionally, said implant comprises a plurality of extensions such that said plurality of extensions define a flared section for said stent. Optionally, said flaring is symmetric. Alternatively or additionally, said flaring has an axis that is at an angle to an axis of said stent. Alternatively or additionally, said flaring comprises a coupling between different extensions such that a flaring angle at one side of the flare compensate for a flare angle at another side of the flare.

In an exemplary embodiment of the invention, said flaring is defined on a side of said stent. Optionally, said flaring has an axis generally perpendicular to an axis of said stent. Alternatively or additionally, said flaring is generally cylindrical.

In an exemplary embodiment of the invention, said stent is a mesh stent. Optionally, said flared section is a mesh.

There is also provided in accordance with an exemplary embodiment of the invention, a method of distorting a medical implant structure having two extensions coupled at a point thereof, comprising:
changing the relative position of two points on said extensions that are distanced from said coupling point;
transforming, using a plurality of pre-defined hinges, tension forces applied by said changing into forces that bend said structure in a plane outside of a plane defined by said changing and by at least a planar portion of said extensions. Optionally, said structure is cylindrical. Optionally, said changing is applied by radially expanding said cylindrical structure. Alternatively or additionally, transforming comprises flaring out said extension to more than 50 degrees relative to an axis of said cylinder. Optionally, said flaring includes a change in angle relative to said axis, along said extensions.

In an exemplary embodiment of the invention, said medical implant is inside a body during said changing and transforming.

There is also provided in accordance with an exemplary embodiment of the invention, a method of implanting a stent, comprising:
conveying a stent to a bifurcation location;
extending at least one advance limiter which is not part of said stent;
advancing said stent until said advance limiter contacts a vessel of said bifurcation of other than a vessel in which said stent is to be implanted; and
expending said advanced stent.

Optionally, extending comprises expanding a mechanical structure. Alternatively, extending comprises inflating an inflatable structure.

There is also provided in accordance with an exemplary embodiment of the invention, a catheter including an advance limiter, comprising:
a catheter adapted to carry a stent thereon; and
an advance limiter configured to selectably extend in a general direction of an axis of said catheter, and away from said catheter. Optionally, said advance limiter is configured to extend in a direction of said stent and extend at least partly past a plane that is perpendicular to an axis of said stent. Alternatively or additionally, said advance limiter comprises a balloon structure. Alternatively or additionally, said advance limiter comprises a mechanically extending structure. Optionally, said advance limiter comprises a self-extending structure. Alternatively, said advance limiter comprises a manually-extending structure.

There is also provided in accordance with an exemplary embodiment of the invention, mesh stent comprising:
a cylindrical body adapted to be inserted in a body and stent a blood vessel; and
a mesh flared section adapted to flare out to more than 90 degrees without tearing of said mesh. Optionally, said flared section comprises a plurality of radially expandable sections and wherein said radially expandable sections each includes a wire section with one or more bends and wherein a length of wire in said sections increases when going in a direction away from a center of said body. Optionally, a number of said bends increases in said direction.

In an exemplary embodiment of the invention, said stent is cut from a sheet or tube.

### BRIEF DESCRIPTION OF THE FIGURES

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIGS. 1A-1D illustrate a method of implanting an ostial stent in accordance with an exemplary embodiment of the invention;

FIG. 2 illustrates advance limiters, in accordance with some exemplary embodiments of the invention;

FIG. 3 is a plan view of an ostial stent in accordance with an exemplary embodiment of the invention;

FIG. 4 is an enlarged view of an ostial end of an ostial stent, as in FIG. 3, in accordance with an exemplary embodiment of the invention;

FIGS. 5 and 6 are cross-sectional views of the ostial portion of the stent of FIG. 4, at two planes of modification of the stent, in accordance with an exemplary embodiment of the invention;

FIG. 7 is a schematic showing of a single flareable element, in accordance with an exemplary embodiment of the invention;

FIGS. 8A-8F show the element of FIG. 7 in various flaring stages, in accordance with an exemplary embodiment of the invention;

FIGS. 9A-9F illustrate schematically various out of plane distortion designs for implants, in accordance with an exemplary embodiment of the invention;

FIGS. 10A-10F illustrate various voids and void series for supporting a distortion mechanism in accordance with an exemplary embodiment of the invention;

FIG. 10G illustrates a flaring element having more than two sets of hinges, in accordance with an exemplary embodiment of the invention;

FIG. 11 illustrates a stent in which the flared section is made of a mesh, in accordance with an exemplary embodiment of the invention; and

FIG. 12 illustrates an ostial stent in accordance with an alternative exemplary embodiment of the invention;

### DETAILED DESCRIPTION OF EXEMPLARY EMBODIMENTS

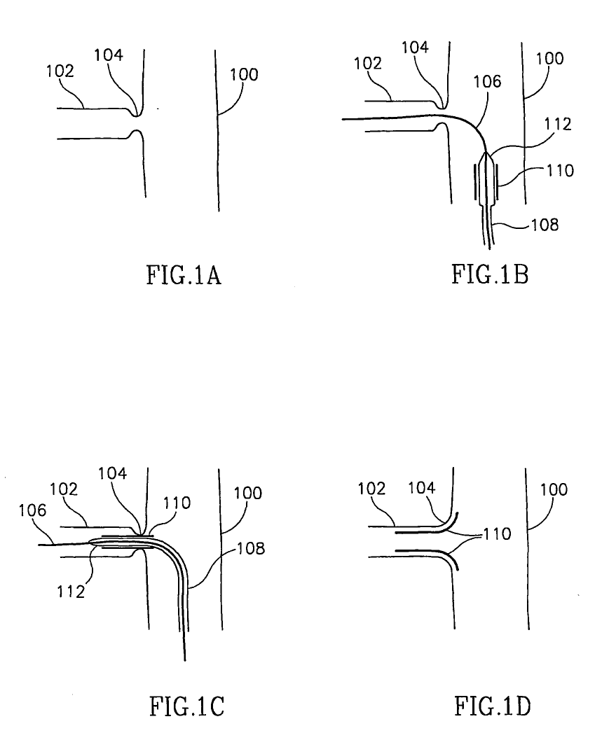

FIGS. 1A-1D illustrate a method of implanting an ostial stent 110 in accordance with an exemplary embodiment of the invention.

A main vessel 100 meets a side vessel 102 at a bifurcation point 104, also known as an ostium. Often, ostium 104 has a reduced diameter, due, for example, to deposits of plaque. A standard treatment is implanting a stent near the ostium. In an exemplary embodiment of the invention, stent 110 is implanted at the ostium and supporting the ostium.

FIG. 1A shows a pre-stenting situation, while a 90 degree angle of branch 102 is shown, the bifurcation angle can be any angle that is found in the body, e.g., between 0 and 90 degrees (for the smaller of the two angles).

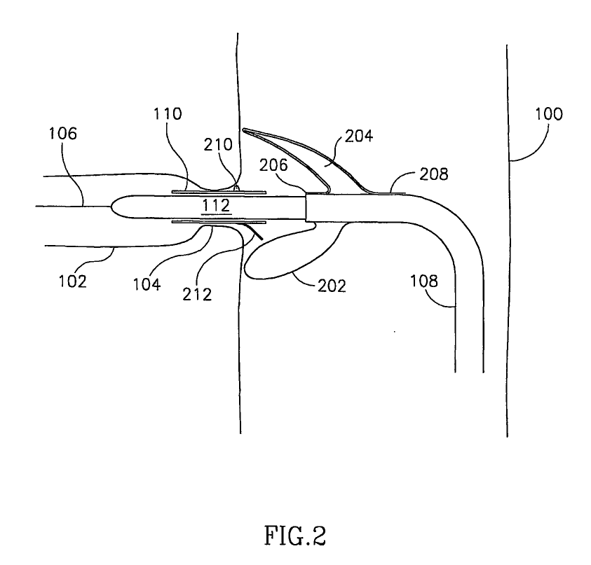

In FIG. 1B, a guide wire 106 is snaked through ostium 104 and a catheter 108, having a balloon 112, on which stent 110 rides, is guided along the guide wire. Other methods of providing stent 110 to the ostium, besides balloon riding and using guidewires may be used, for example as known in the art. Alternatively or additionally, some local treatment may be performed prior to providing stent 110, for example, expansion of a balloon in the ostium. Stent 110 may be of various types. In some embodiments of the invention a self flaring stent as described below is provided. In other embodiments, for example in conjunction with a limiter as shown in FIG. 2, a standard or prior art flareable stent may be provided.

In FIG. 1C, stent 110 is aligned in the ostium, for example using methods described in FIG. 2 and/or using fluoroscopic or other imaging techniques. Balloon 112 is then inflated. Balloon 112 maybe, for example, a standard type balloon, an elongate balloon or a specialized balloon, for example as described below.

In FIG. 1D, stent 110 is already expanded to engage vessel 102 and flared, to cover ostium 104. The guidewire and catheter 108 have been removed.

As noted above, one potential problem is correct axial placement of the ostial stent in ostium 104. In an exemplary embodiment of the invention, correct axial placement is provided by limiting the advance of stent 110, for example, by providing a flaring on stent 110 and/or providing a limiting mechanism on catheter 108. FIG. 2 illustrates advance limiters, in accordance with some exemplary embodiments of the invention. Several different mechanisms are shown on the same catheter. In practice, typically only one of these is used in a particular case.

In one exemplary embodiment of the invention, a balloon extension 202 is provided on catheter 108. When balloon 202 is expanded, the effective diameter of catheter 108 is greater than that of vessel 102, thus limiting the advance of catheter 108 and stent 110. Balloon 202 is shown as a finger, of which a plurality may be provided. Optionally, the finger contacts the wall of vessel 100 past a point of flaring of stent 110, so that it does not interfere with its flaring. Balloon 202 is optionally inflated by the same inflation lumen as balloon 112, as generally lower inflation pressures are needed. Alternatively, a skirt shaped balloon is provided. Alternatively, the balloon may be spherical. Optionally, the balloons 202 (for a multi-finger embodiment) are not all the same length, defining a desired approach angle to side vessel 102.

Optionally, the catheter tends to align its orientation with respect to the side vessel. In an exemplary embodiment of the invention, balloon 202 is a single balloon with a slot defined so that the catheter naturally is urged to rotate and align the slot with the balloon. In this way, the stent may have its oblique orientation matched with a vessel's oblique orientation. A similar modification may be provided for other balloon schemes.

In an alternative embodiment of the invention, a mechanical mechanism is provided as an advance limiter. For example, an axially slotted tube 208 is provided on catheter 108. a forward end 206 of tube 208 is restrained, for example by attachment to catheter 108 or using an inner tube (not shown) that is held outside the body. When tube 208 is advanced, the slots define one or more fingers 204.

In an alternative embodiment of the invention, the axial alignment is visual. In one example, a radio-opaque fiduciary mark 210 is provided on stent 110 and a fluoroscope (for example) is used, to determine when mark 210 is aligned correctly. In case of an oblique connection, a ring of radio-opaque material or a plurality of marks is optionally provided.

In an alternative embodiment of the invention, stent 110 includes a flaring section 212 that is flared (and/or expanded radially) prior to complete radial expansion of stent 110, so stent 110 can still be advanced into side vessel 102. In the case of a super elastic, elastic or shape memory stent, a flareable outer tube (e.g., like tube 208) may be provided, to allow the stent to flare before being axially advanced.

In an exemplary embodiment of the invention, stent 110 is partially inflated, so that section 212 flares. One possible way of partial flaring is using a balloon that expands first or only adjacent flared section 212, thereby flaring it before expanding stent 110 an undue amount. Alternatively or additionally, flared section 212 may be weakened or be constructed to be weaker, so that it expands first. Alternatively or additionally, flared section 212 may include an elastic, shape memory or super elastic component, which allows it to flare without and balloon expansion. Alternatively or additionally, stent 110 is a self-flaring stent as described below, in which partial expansion of stent 110 flares section 212 partially or completely.

Once sufficient flaring is provided, for example being visible on a fluoroscope (e.g., using radio-opaque markets on stent 110 if necessary), the stent is advanced until the flared section 212 is correctly in contact with ostium 104 and then the expansion and, possibly flaring of stent 110 are completed.

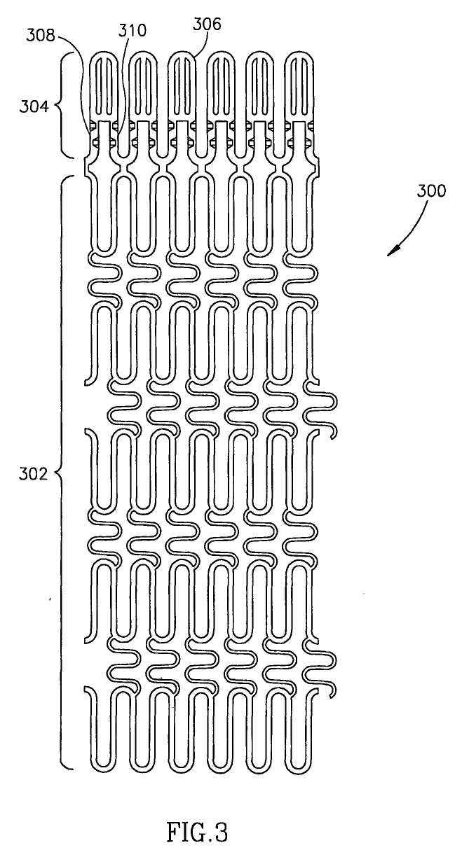

FIG. 3 is a plan view of an ostial stent 300 in accordance with an exemplary embodiment of the invention. A first part of stent 300 is a cylindrical section 302, which may be, for example, of any construction known in the art for radially expandable stents or other types of stents. A second part of stent 300 is a flaring section 304. The two parts may be attached using means known in the art, for example welding, mechanical attachment, soldering and gluing. Alternatively or additionally, they may be formed of a single piece, for example a tube or a sheet. In an exemplary embodiment of the invention, section 304 comprises a plurality of flare segments 306 that are attached to cylindrical section 302 by pairs of fingers 308 and 310. In some embodiments, more than two fingers are provided, for example three or more. In an exemplary embodiment of the invention, fingers 308 are mirror-symmetric with respect to the axis of stent 300 and include a plurality of hinge locations, described below. In some embodiments of the invention, alternatively or additionally, one or more of the fingers is replaced by a differently formed element, for example a rigid strut or a finger with a different number of hinge locations and/or hinges of different types than those of the other finger.

Optionally, one or more radio opaque markers or bands are provided on the stent, for example, at the tip of the flaring section, at the junction between the flaring section and the body, at the bend location and/or along the body.

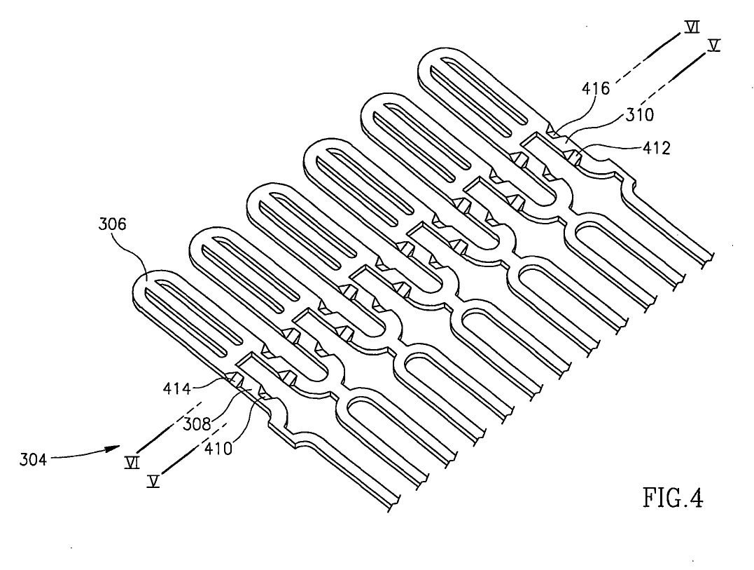

FIG. 4 is an expanded view of flaring section 304, in accordance with an exemplary embodiment of the invention. The figure is shown at an oblique angle, to better emphasize the hinges. As shown, finger 308 has two hinges, 410 and 414 defined on it and finger 310 has hinges 412 and 416 defined on it. As shown, the hinges are formed by oblique cuts, however, other methods of forming hinges may be used, for example as described below. In this embodiment, hinges 410 and 412 face each other and tend to support bending of the fingers towards each other and out of the plane towards an observer. It should be noted that the hinges are activated by fingers 308 and 310 being pulled away from each other, by radial expansion of stent 300. Hinges 414 and 416 face away from each other and towards the observer. As will be explained below, this design on hinges has a net effect of bending segment 306 towards an observer, while accommodating at least some stresses induced by this distortion and/or supporting the displacement of the fingers from each other, at their base.

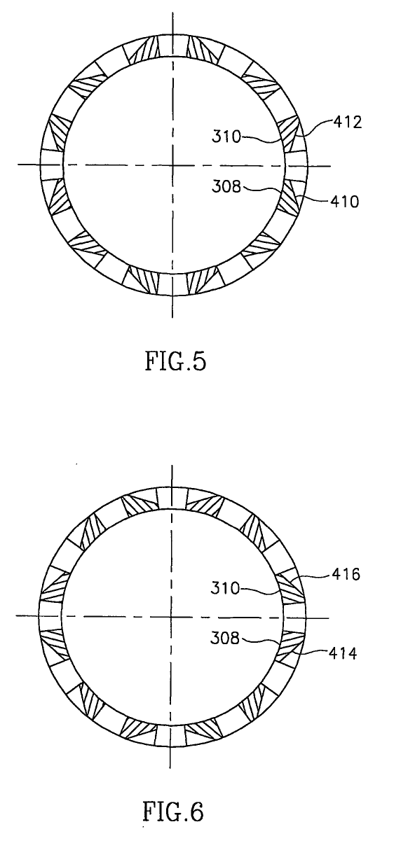

FIGS. 5 and 6 are cross-sectional views of the ostial portion of the stent of FIG. 4, at two planes of modification of the stent, in accordance with an exemplary embodiment of the invention. FIG. 5 shows a cut through lower hinges 410 and 412. FIG. 6 shows a cut through upper hinges 414 and 416.

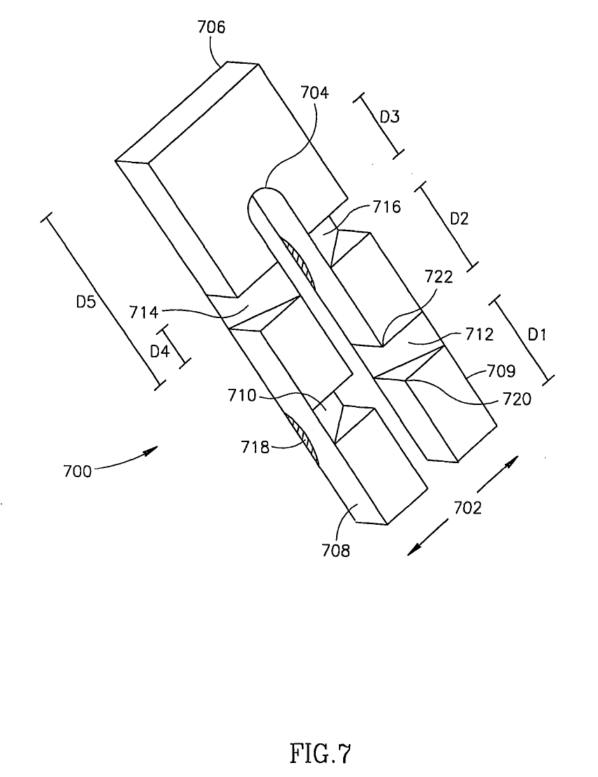

FIG. 7 is a schematic showing of a single flareable element 700, in accordance with an exemplary embodiment of the invention. Element 700 comprises two fingers, 708 and 709, that move apart as shown by arrow 702. The fingers are attached at one end by an apex 706. As in FIG. 6, finger 708 and 709 define mirror images of hinges, bottom hinges 710 and 712 and upper hinges 714 and 716. While the hinges are shown as obliquely cut slots, other forms may be provided as well. Optionally, a back section 718 of a hinge is removed, to reduce a resistance to bending offered by that part of the hinge, especially in materials resistant to elongation, and/or to prevent tearing or other distortion of the hinge.

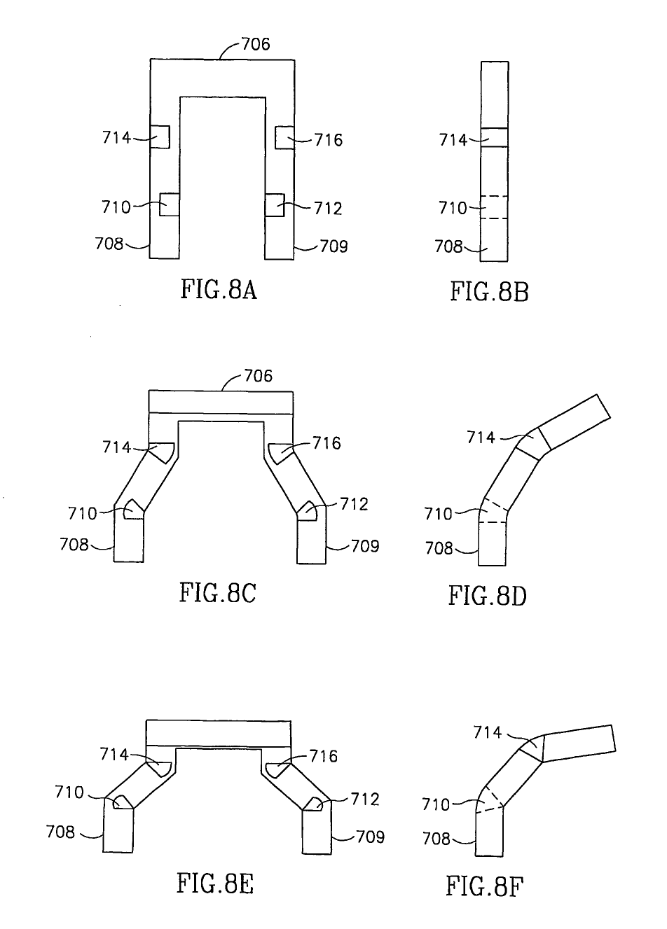

FIGS. 8A-8F show flaring element 700 in various flaring stages, in accordance with an exemplary embodiment of the invention. FIG. 8A is a front view similar to FIG. 7. FIG. 8B is a side view of FIG. 8A.

FIGS. 8C and 8D show element 700 after the bases of fingers 708 and 709 have been displaced radially a small amount. Apex 706 prevents the tips ends of fingers 708 and 709 from also moving apart, which results in strain in element 700. If apex 706 and especially a bifurcation point 704 thereof are rigid enough, the strain must be relieved by distortion of fingers 708 and 709. If the attachment of the fingers at their base is also rigid, the preferred distortion point is defined by the hinges. However, rather than giving in the direction of the strain, the hinges add an out of plane component to the distortion.

In FIGS. 8E and 8F, the fingers have been moved further apart. In an exemplary embodiment of the invention, as shown, the hinges optionally define a maximum bend allowed by the hinges, for example, hinge 712 reaches its maximal bend when its two end points 720 and 722 meet. In FIG. 5, these points are cut in the shape of a plane, to ensure better contact and/or less distortion when they meet. As shown in FIGS. 8E and 8F, flaring element 700 reaches its maximal bend out of the plane at this point. Possibly, if the stent is expanded further and fingers 708 and 709 move further apart, the stress will cause distortion at points of the stent other than the hinges. In one example, apex 706 gives at bifurcation 704. Alternatively or additionally, the points of attachment at the bases of the fingers give or the areas below hinges 710 and 712.

In some embodiments of the invention, the distortion of the hinges includes a plastic deformation that is not reversible. In others, the deformation is elastic and/or reversible. In addition, apex 706 may distort a small amount, this distortion may also include a cupping, along an axis that continues bifurcation point 704 along the direction of the fingers. Possibly, this cupping accommodates twist behavior of the fingers. In general, some distortion of apex 706 is expected due to its function in coupling the distortion of the two fingers and its accommodation of opposing forces from the fingers. However, such distortion is generally negligible with regard to the function of element 700.

Referring back to FIG. 7, various dimensions of element 700 may be varied, to achieve various effects, in accordance with exemplary embodiments of the invention. D3, for example, the distance between the top hinge and bifurcation point 704 (and related to it, the distance between the fingers), may define the lever applied towards distorting apex 706. In general, the smaller the better, if no distortion is desired.

D2, the distance between upper and lower hinges, is the part of element 700 that is neither at the final flaring angle nor at the stent plane. This length may depend on the application, for example, longer for oblique angles between blood vessels. In addition, the shorter D2 is, the greater the flaring effect of a given radial displacement of the fingers. The flaring effect is also determined, at least in part by the angle of the hinges relative to the plane.

D1, the distance between the base of the fingers and the lower hinges, may depend, for example, on a desire to have the attachment at the base distort when the flaring is completed and radial expansion continues. The longer D1 is, the greater the lever for distortion.

D4, for example, may help determine the strain on the material. Making D4 larger distributes the strain over a greater area.

D5, for example, may define, the extent of the flaring. In addition, suitable selection of D1-D3 may be used to define a target shape of the stent for a certain expected expansion situation.

While shown mirror-symmetrical, fingers 708 and 709 need not be symmetric. In one example, finger 709 is replaced by a wire, which can freely distort. This will limit the movement of the tip of finger 708, but since it will not bend exactly at hinge points, the distortion will be different. This may find use, for example, by an aperture being defined perpendicular to the stent axis and between the wire and the finger. Asymmetrical fingers can also be used to support twisting of fingers (relative to themselves and/or the device plane), which may be useful, for example, if the fingers include barbs that are thus twisted to engage or disengage adjacent tissues or structures.

In another example, finger 709 has hinge 712 closer to its base than hinge 710 is to the base of finger 708, this will cause bending to the side of the flaring. In another example, the fingers have different numbers of hinges and/or different types, for example, finger 709 has only a centered non-oblique hinge (e.g., parallel or perpendicular to the plane of the finger).

While two hinges are shown, a greater number of hinges may be used as well, for example, three or four.

As noted elsewhere in the application, varying the hinge locations, lengths D1-D5 and/or hinge angles may be used to vary the flaring properties, for example, the ratio of flaring to expansion, flare diameter and/or the final flaring, optionally within a single stent, to provide an asymmetrically flaring stent.

Alternatively or additionally to setting hinge locations, in an exemplary embodiment of the invention, a preferred hinge angle is set by a design process. Alternatively, hinges with a range of possible bending directions are provided. In an exemplary embodiment of the invention, the design process is as follows. First, a desired flaring end result is defined and then a search is made, for example, using methods known in the art, for device parameters that will match that result. In one example, a search is made on the space defined by the parameters of hinge location, assuming that the hinges are can bend in any direction. Then a preferred hinge direction is determined by selecting a hinge direction in which the strain on the hinge is minimal, for example along the bending process and/or at some point of the bending process. Other selection methods may be used as well, for example, searching for a hinge angle at which the stress is always below a threshold. In some cases, an analysis is made for the process of bending as well, for example to ensure that the force relative to the moment of inertia of the hinge is always sufficiently greater than at other parts of the stent to prevent undesired distortion. In an exemplary embodiment of the invention, vector analysis is used to assist in designing the flaring section.

Referring back to FIG. 7, an example of a non-stent and non-cylindrical device is a clip, formed by attaching two elements 700 base to base. When the fingers are pried apart at the base, the edges bend and can meet (depending on the parameters chosen). To remove the clip, the fingers are approximated at the base and the clip unbends. This is also an example that shows that the out of plane distortions can be retracted. This may find use if a bifurcation stent is to be removed and the stent includes a contraction mechanism, for example cooling a shape memory stent. This can be used for example to anchor an electrode.

It should be noted that the above described structure can bend when the two “fingers” are moved towards each other. For example, in the structure of FIG. 7, the spacing between the fingers is made bigger and the hinges are arranged opposite of what is shown. Then, bringing the fingers towards each other, will have the effect of bending the fingers out of plane. This bending is limited by the distance, if the fingers are configured to abut each other and then stop. Alternatively, the fingers may slide past each other.

It should also be noted that moving the fingers can also straighten the fingers, for example, if the fingers are pre-bent, at one or more locations thereof.

It should also be noted that in some designs, if the fingers encounter resistance to bending, this resistance may be transmitted back to cause resistance to moving apart of the fingers. In a stent, this may cause a greater expansion of a different part of the stent, with greater flaring there. This mechanism may also be used, for some flaring angles and stent designs, for collapsing a stent incorporating the flaring, by compressing the flaring.

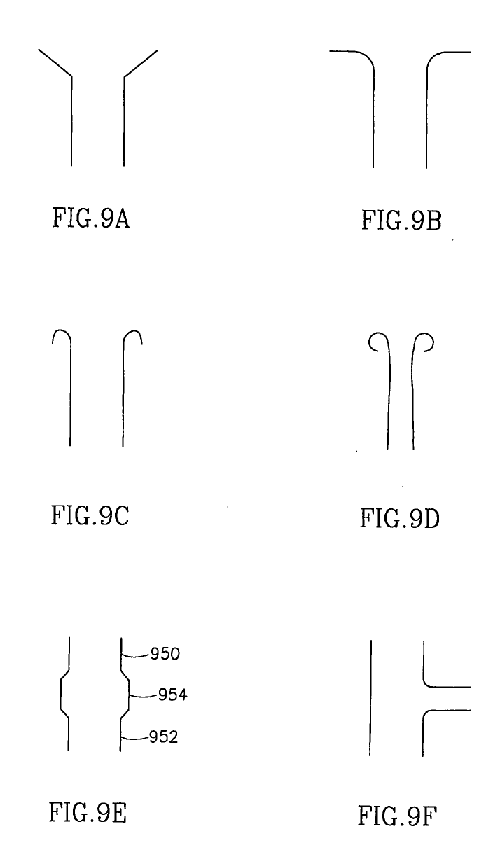

FIGS. 9A-9F illustrate schematically various out of plane distortion designs for deployed implants, in accordance with an exemplary embodiment of the invention.

FIG. 9A shows a flaring smaller than 90 degrees, for example, 89 80, 70, 50, 45, 30 or any intermediate or smaller number of degrees. These angles are exemplary of some types of bifurcation in the vascular system.

FIG. 9B shows a flaring of 90 degrees. This may be useful, for example, in the aorta, for cardiac arteries.

FIG. 9C shows a flaring greater than 90 degrees, for example, 91, 100, 110, 130, 140, 150 or any intermediate or greater angle. These are also exemplary values for bifurcation and also, in some cases, provided better anchoring by forcing the flared portions into the tissue. In some cases, more than two hinges per finger may be required, for example, three or four. The examples of FIGS. 9A-9C may be combined to provide a stent with an oblique flaring. In an exemplary embodiment of the invention, a radio-opaque marker is used so that the orientation of the stent can be determined from outside the body.

FIG. 9D shows an example where the flaring curls on itself. This may be useful, for example, when anchoring to the end of a tubular element or to prevent sharp edges from protruding away from the stent.

FIG. 9E shows an example where a stent is expanded at its center, for example, to assist in anchoring. In an exemplary embodiment of the invention, the widening comprises two rings of hinge areas 950 and 952, facing each other. In an exemplary embodiment of the invention, a section 954 between the rings comprises wave segments, which can axially elongate to compensate for axial shortening caused by flaring. In one implementation, the upper hinges of the hinge areas are on the opposite face of the stent, so that section 954 remains is in a plane parallel to the stent plane. This structure may also be used for an end of a stent, for example to define a cone or two diameter stent.

While the plane of the hinges is shown to be perpendicular to the stent axis, it can also be oblique or even not define a plane, for example defining two planes or being irregular, for example as shown below This may find use for some configuration of desired flare angle and flare profile.

FIG. 9F shows an example of a stent having side opening. In an exemplary embodiment of the invention, this opening is created by a ring of flare areas defined on a side wall of a stent, instead of around its circumference. The different flaring elements may use different degrees of response to expansion and final position, for example to compensate for the different amounts of expansion and angles at different stent locations. In an exemplary embodiment of the invention, this stent design is used for a bifurcation stent, for defining an opening into a side vessel and/or for assisting in axial and/or rotational alignment of the stent, if it flares out sufficiently prior to complete expansion. It should be appreciated that in some stents, radial expansion does not affect axial dimensions. Thus, a flaring element attached to the stent at 9 o'clock of the side opening (assuming the stent is pointed at 12 o'clock) may experience no separation of its fingers and thus no flaring. In an exemplary embodiment of the invention, the two fingers are attached to different angular sections of the stent (e.g., at same or different axial positions along the stent), which move apart during expansion. Alternatively or additionally, the fingers are attached obliquely, e.g., at 8 o'clock for a 9 o'clock flaring element.

In some embodiments of the invention, the flared sections are pre-bent, for example along the device axis and opposite to the curve of the stent. This may be used to accommodate curvature of a surface that the flaring section is in contact with.

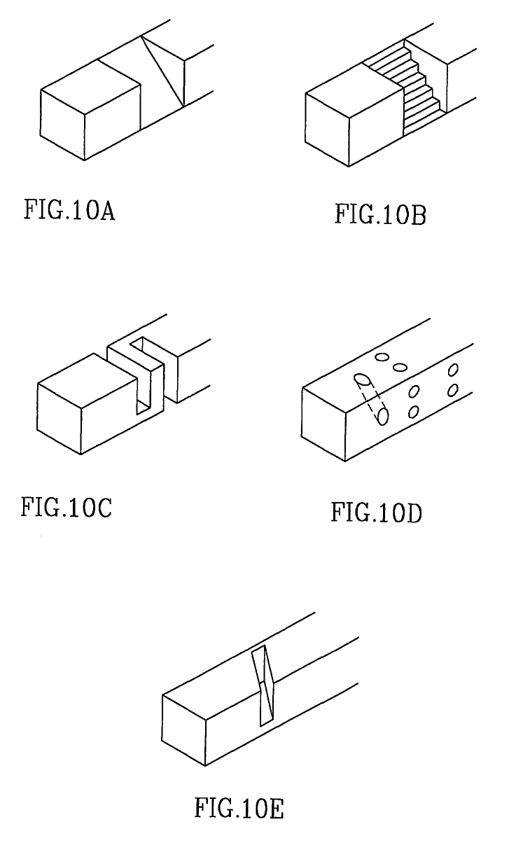

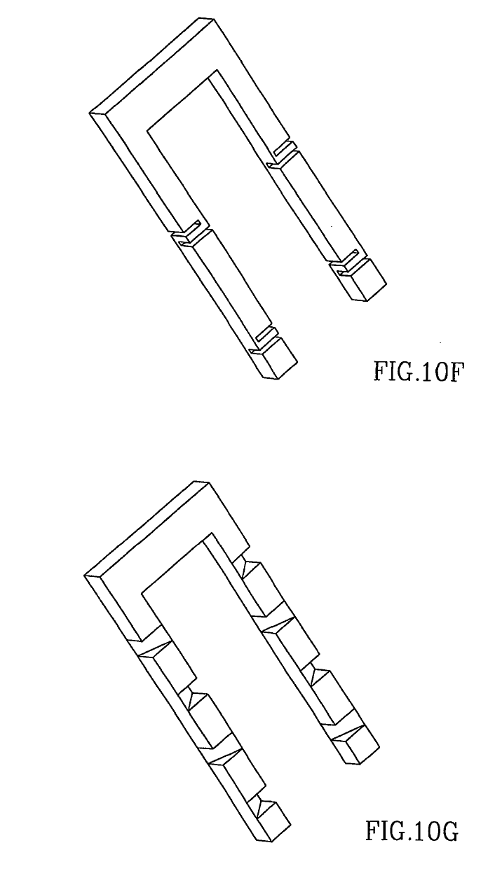

FIGS. 10A-10F illustrate various voids and void series for supporting a distortion mechanism in accordance with exemplary embodiments of the invention.

FIG. 10A shows a simple wedge hinge, where a triangular section of material is removed from a stent, at an oblique angle to the stent plane. In this design, the moment of inertia of a unmodified part of the stent is I=bh^{3/}12, where b is the width and h the thickness of an element in which a hinge is formed. Assuming b=h, we get I=b⁴/12. Assuming the hinge is a simple diagonal cut, the moment of inertia is I=b⁴/101.8, which indicates that substantially all the forces (and thus the distortion) will be focused at the hinge. In some embodiments of the invention, the narrower the slot, the more efficient the force redirection (e.g., less stress outside the hinge). Optionally, sharp edges, if any, are smoothed or cut away, for example, to prevent potential damage to a nearby blood vessel, balloon and/or other natural or unnatural structure and/or to prevent stress concentrations in the structure.

Various oblique angles may be provided for the hinges, for example, the hinge may have a preferred bending direction that is between 10 and 90 degrees relative to a device plane. Other angles may be provided as well, for example, greater than 20, 30, 40, 60 degrees, or smaller than 70 degrees, or any intermediate values. The hinge may also be oblique to an axis of a structural element in which it is defined, for example having an angle of less than 90, 80, 70, 60, 50 or any intermediate or smaller angle between a plane of the hinge bending and the axis of the structural element.

In some cases, a smooth cut may be more difficult to make. Instead, a stepped slot may be made, for example, as shown in FIG. 10B.

In some case, an oblique cut is replaced by a series of two or more non-oblique cuts, one in the stent plane and one perpendicular to it. This is shown for example in FIGS. 10C and 10F (a complete exemplary flaring element).

Alternatively or additionally to cuts, hinge definition may be by drilling holes, for example as shown in FIG. 10D. The holes may be oblique or perpendicular and/or in more than one plane. The various types of hinge definition methods may be combined, as well. For example, a hinge may be defined using both slots and holes. Alternatively or additionally, holes may be drilled through a hinge to reduce stress while bending and/or allow material flow.

In some embodiments of the invention, a hinge is defined using a cut that is oblique to the axis of the cut element and thus oblique to one or both non-end planes of the element, for example as shown in FIG. I E. While mainly straight cuts are shown, cuts may be curved, for example, scalloped and crescent shaped voids that are removed.

Alternatively or additionally to using cuts and voids to weaken an element and define a joint, various metallurgical treatments may be used, for example, annealing and acid baths. Cold working and hardening may be selectively applied to non-hinge areas. In some cases, annealing may be used in addition to increase the plastic deformation range (e.g., elongation) of a hinge.

In an exemplary embodiment of the invention, stent 300 is manufactured from a tube that is cut with a laser a water jet, e-beam, plasma beam or acid etching as known in the art, or formed of a plate that is rolled and welded into a cylinder. Optionally, the same methods are used, albeit at an angle, to form the hinges. In chemical etching means, a varying thickness mask may be used on the slots. Varying thickness slots may be cut, for example, using a timed perpendicular cut rather than an oblique cut. However, other manufacturing methods may be used as well.

Alternatively or additionally, the hinges are constructed, for example, by adding a layer, for example by depositing, welding or bonding. Stiffness of various parts of the stent (e.g., at the attachment of fingers 708, 709 to the stent) may also be varied, for example by using non-straight cuts.

In an exemplary embodiment of the invention, the material used can plastically deform, for example, SS 316, titanium or titanium alloys. Alternatively or additionally, a super elastic stent may be provided. For example, all of the flared section may be super elastic or shape memory or all the stent may be super elastic or shape memory. Optionally, the flaring starts out using a small amount of elastic, super elastic or shape memory effect, for example, to assist in placement of the stent before its expansion (e.g., when an over tube is retracted), by the hinges are plastically deformed. In an exemplary embodiment of the invention, the stent is designed so that the maximum flaring is achieved before the stent is completely expanded. This is optionally useful for preventing spring back from undoing the flaring and/or for preventing the need to over-flare.

FIG. 10G illustrates a flaring element comprising more than two sets of hinges. In this case, four. In an exemplary embodiment of the invention, large numbers of hinges are used to provide a significant roll back.

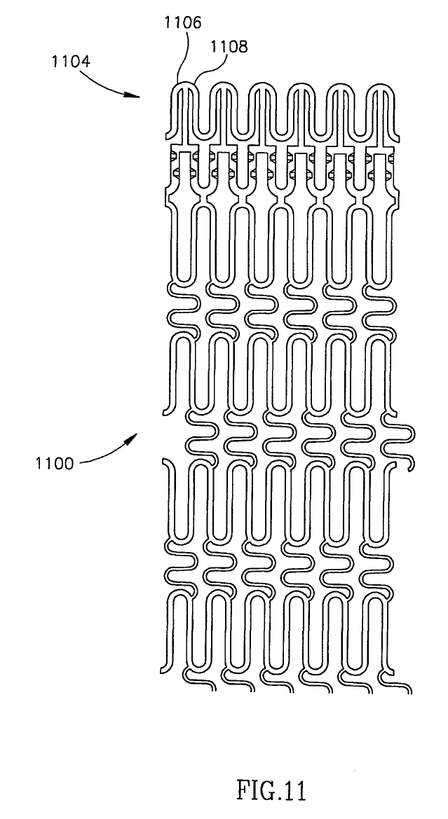

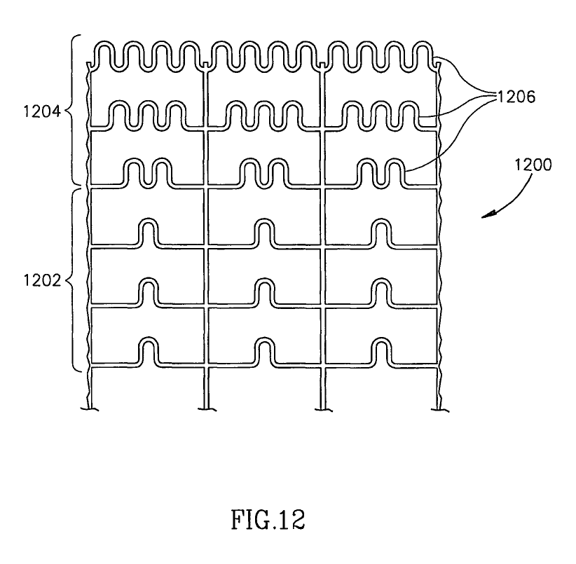

FIG. 11 illustrates a stent 1100 in which a flared section 1104 is made in the form of an array of closed cells, similar to a mesh, in accordance with an exemplary embodiment of the invention. In this embodiment, a plurality of flaring elements 1106 are interconnected using a plurality of wires or other elements 1108. Depending on the relative length of elements 1108 and the flaring design of section 1104, elements 1108 may serve to limit the maximal flaring and/or couple the flaring of one flaring element with another. In an exemplary embodiment of the invention, by limiting the total amount of flaring to have a certain diameter, if one side of the stent cannot flare as much (e.g., it contacts vascular tissue) the other side can flare more. Optionally, elements 1108 includes an additional bend, to allow for a greater length of the element in the design shown. Alternatively or additionally, elements 1108 may originally point away from the stent, in which direction the stent does not limit their length prior to deployment. Instead of a single layer of wire, two axially spaced wires may be provided. In some exemplary embodiments of the invention, when a mesh is used in a flaring portion, a significant amount of free wire is provided, for example in the shape of folding as shown in FIG. 12, below, to allow for the increased diameter of the flared section.

FIG. 12 illustrates an ostial stent 1200, in plan view in accordance with an alternative exemplary embodiment of the invention. This stent includes a cylindrical section 1202, which may be for example, of any type known in the art. A flaring section 1204, includes a greater amount of radial expansion ability in a plurality of radial links 1206. Optionally, the flaring shape is implemented by providing an increase (along the axis) of the available radial expansion ability. In an exemplary embodiment of the invention, during deployment, a regular balloon is used, optionally one that is extends significantly beyond the flaring section. In an exemplary embodiment of the invention, the flaring section is made weaker, for example, by selective thining or heat treatment (this may also be applied to a uniformly designed stent), so that as the balloon inflated, the flaring section will expand first. Alternatively or additionally, the balloon is designed to expand first adjacent the flaring section, for example, be comprised of two compartments. In an exemplary embodiment of the invention, the uncovered part of the balloon expands first, assisting in rolling open the flared section. This rolling may also be used in the previously described embodiments, for example, to assist in initial placement of the stent. It should be noted that the design of FIG. 12 may also be applied to that of FIG. 11, for example to provide multiple, axially separated, rings in a flared section. It should also be noted that one or more axial segments of the flared section may be unmodified (e.g., lacking hinges) or have hinges with a bending direction that does not cause out of plane motion, for example, all the hinges with a bending direction perpendicular to the device plane. Alternatively, the flaring out is achieved using a balloon which expands the flaring section directly and not indirectly as described for example in FIG. 3.

In some embodiments of the invention, a stent or expanding structure as described above is used as part of a stent-graft or a stented graft, for example with a graft mounted on the non-flaring and/or flaring parts of the stent. Alternatively or additionally, such a stent is used for a prostate, with the flaring used for anchoring in the bladder, and, possibly a second, smaller flaring or expansion for anchoring in the urethra.

It will be appreciated that the above described methods and mechanisms for bending out of plane may be varied in many ways, including, for example, the exact materials used for the devices, the direction of bending, the type of hinges and/or their numbers. Further, in the mechanical embodiments, the location of various elements may be switched, without exceeding the spirit of the disclosure, for example, switching the moving elements for non- moving elements where relative motion is required. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of the above features, from different described embodiments are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered as necessarily limiting the invention in its broadest aspect to only those forms, for example, where a circular lumen is shown, in other embodiments an oval lumen may be used.

Also within the scope of the invention are surgical kits which include sets of medical devices suitable for making a single or several stent applications. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A deformable medical implant, comprising:
a body defining at least two anchor points, which body is adapted to be deformed so that the two anchor points are moved relative to each other;
at least two elongate extensions, each extension fixed to one anchor point;
a bridge coupling at least two of said extensions to each other; and
at least two hinges defined on at least one of said extensions, two of said at least two hinges having different preferred bending directions and being defined on one extension.

2. An implant according to claim 1, wherein said two elongate extensions each comprise a plurality of hinges.

3. An implant according to claim 2, wherein the hinges on said one elongate extension are a mirror of the hinges on the other, coupled, extension.

4. An implant according to claim 2, wherein the hinges on said one elongate extension have different axial locations than corresponding hinges a second, coupled, elongate extension.

5. An implant according to claim 2, wherein at least one of the hinges on said one elongate extension has a hinge bending direction different from corresponding hinges a second, coupled, elongate extension.

6. An implant according to claim 2, wherein at least one of the hinges on said one elongate extension has a resistance to bending different from corresponding hinges a second, coupled, elongate extension.

7. An implant according to claim 1, wherein only one of said at least two elongate extensions comprises a plurality of hinges.

8. An implant according to claim 1, wherein at least two of said plurality of hinges have bending axes that are oblique to a device plane of said body, said device plane being a substantially two-dimensional mathematical surface conforming to the general geometry of the device.

9. An implant according to claim 1, wherein at least one of said plurality of hinges has a preferred bending direction in a device plane of said body, said device plane being a substantially two-dimensional mathematical surface conforming to the general geometry of the device.

10. An implant according to claim 9, wherein at least one of said plurality of hinges has a preferred bending direction perpendicular to a device plane of said body, said device plane being a substantially two-dimensional mathematical surface conforming to the general geometry of the device.

11. An implant according to claim 1, wherein said hinges are arranged to cooperate with said bridge to bend said extensions in a direction including a component perpendicular to a device plane of said body, when said anchor points are moved apart, said device plane being a substantially two-dimensional mathematical surface conforming to the general geometry of the device.

12. An implant according to claim 11, wherein said hinges are arranged to bend at least one of said extensions at at least two points, in different directions.

13. An implant according to claim 11, wherein said hinges are arranged to bend said extensions at least 45 degrees away from said device plane.

14. An implant according to claim 11, wherein said hinges are arranged to bend said extensions at least 80 degrees away from said device plane.

15. An implant according to claim 11, wherein said hinges are arranged to bend said extensions at least 90 degrees away from said device plane.

16. An implant according to claim 11, wherein said hinges are arranged to bend said extensions at least 120 degrees away from said device plane.

17. An implant according to claim 1, wherein at least one of said hinges comprises cuts in said extension.

18. An implant according to claim 1, wherein at least one of said hinges comprises a weakening in a position along said extension.

19. An implant according to claim 1, wherein at least one of said hinges comprises a bore in said extension.

20. An implant according to claim 1, wherein said extensions extend axially away from said body, prior to moving apart of said anchor points.

21. An implant according to claim 1, wherein said extensions extend axially towards said body, prior to moving apart of said anchor points.

22. An implant according to claim 1, wherein said bridge is defined at an end of said extensions.

23. An implant according to claim 1, wherein said bridge is deformable.

24. An implant according to claim 23, wherein said bridge is more resistant to bending than said hinges.

25. An implant according to claim 1, wherein said hinges are plastically deformable.

26. An implant according to claim 1, wherein said plurality of hinges comprise at least three hinges on a single extension.

27. An implant according to claim 1, wherein said body is cylindrical.

28. An implant according to claim 1, wherein said implant is adapted for implanting in a blood vessel.

29. An implant according to claim 1, wherein said implant is a stent.

30. An implant according to claim 29, comprising a plurality of extensions such that said plurality of extensions define a flared section for said stent.

31. An implant according to claim 30, wherein said flaring is symmetric.

32. An implant according to claim 30, wherein said flaring has an axis that is at an angle to an axis of said stent.

33. An implant according to claim 30, wherein said flaring comprises a coupling between different extensions such that a flaring angle at one side of the flare compensate for a flare angle at another side of the flare.

34. An implant according to claim 30, wherein said flaring is defined on a side of said stent.

35. An implant according to claim 34, wherein said flaring has an axis generally perpendicular to an axis of said stent.

36. An implant according to claim 34, wherein said flaring is generally cylindrical.

37. An implant according to claim 1, wherein said stent is a mesh stent.

38. An implant according to claim 37, wherein said flared section is a mesh.

39. A method of distorting a medical implant structure having two extensions coupled at a point thereof, comprising:
changing the relative position of two points on said extensions that are distanced from said coupling point;
transforming, using a plurality of pre-defined hinges, tension forces applied by said changing into forces that bend said structure in a plane outside of a plane defined by said changing and by at least a planar portion of said extensions.

40. A method according to claim 39, wherein said structure is cylindrical.

41. A method according to claim 40, wherein said changing is applied by radially expanding said cylindrical structure.

42. A method according to claim 40, wherein transforming comprises flaring out said extension to more than 50 degrees relative to an axis of said cylinder.

43. A method according to claim 42, wherein said flaring includes a change in angle relative to said axis, along said extensions.

44-57. (canceled)
